(19) United States
(12) Patent Application Publication
(10) Pub. No.: US 2022/0026744 A1
(43) Pub. Date: Jan. 27, 2022

(54) Aromatherapy Headwear Apparatus

(71) Applicant: Annette Hutchins, Pt Orchard, WA (US)

(72) Inventor: Annette Hutchins, Pt Orchard, WA (US)

(21) Appl. No.: 16/935,759

(22) Filed: Jul. 22, 2020

(51) Int. Cl. G02C 11/00; A61L 9/12

(52) U.S. Cl. CPC G02C 11/00; A61L 9/12

(57) ABSTRACT

An aromatherapy headwear apparatus for handsfree aromatherapy application includes a glasses frame including a rim portion, a bridge portion coupled to the rim portion, and a pair of arm portions coupled to the rim portion. A pair of lenses is coupled to the rim portion of the glasses frame. A diffuser holder is coupled to an inner side of the glasses frame. An aroma diffuser is selectively engageable with the diffuser holder. The aroma diffuser holds and dispenses an essential oil.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to aromatherapy devices and more particularly pertains to a new aromatherapy device for handsfree aromatherapy application.

### (2) Description of Related Art Including Information Disclosed Under 37 CFR 1.97 and 1.98

The prior art relates to aromatherapy devices. Existing devices allow for glasses and other headwear to secrete perfumes. These devices serve to disperse a scent to other people nearby rather than for the wearer. Existing devices do not allow aromatherapy oils to make direct contact with the forehead and also lack a removable aroma diffuser.

### BRIEF SUMMARY OF THE INVENTION

An embodiment of the disclosure meets the needs presented above by generally comprising a glasses frame including a rim portion, a bridge portion coupled to the rim portion, and a pair of arm portions coupled to the rim portion. A pair of lenses is coupled to the rim portion of the glasses frame. A diffuser holder is coupled to an inner side of the glasses frame. An aroma diffuser is selectively engageable with the diffuser holder. The aroma diffuser holds and dispenses an essential oil.

Another embodiment of the disclosure generally comprises a headband having a headband inside and a headband outside. A diffuser holder is coupled to the headband inside. An aroma diffuser is selectively engageable with the diffuser holder. The aroma diffuser holds and dispenses an essential oil.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is an isometric view of an aromatherapy headwear apparatus according to an embodiment of the disclosure.

FIG. 2 is a detail view of an embodiment of the disclosure.

FIG. 3 is a rear elevation view of an embodiment of the disclosure.

FIG. 4 is a side elevation view of an embodiment of the disclosure.

FIG. 5 is an isometric view of an embodiment of the disclosure.

FIG. 6 is an isometric view of an embodiment of the disclosure.

### DETAILED DESCRIPTION OF THE INVENTION

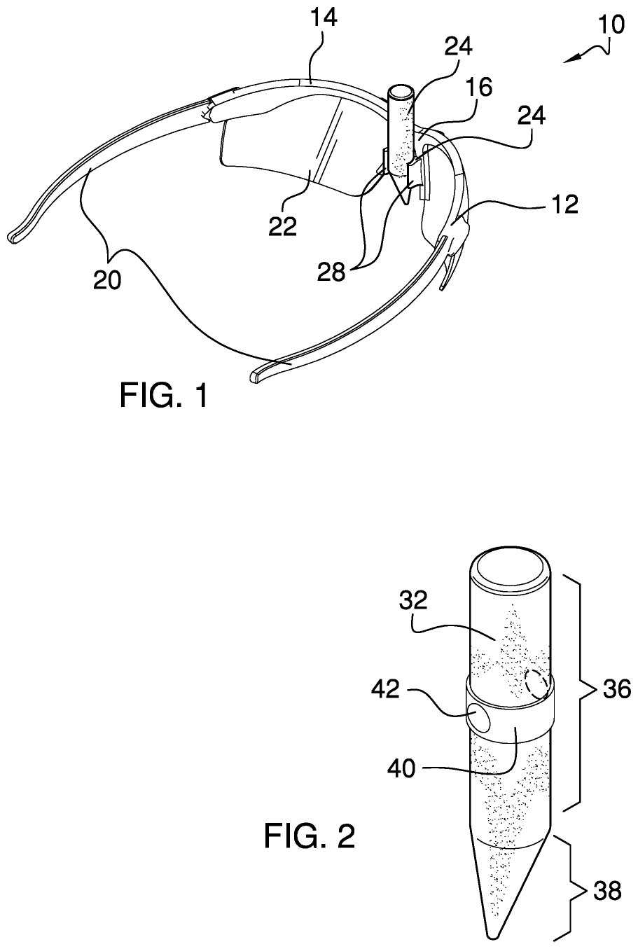

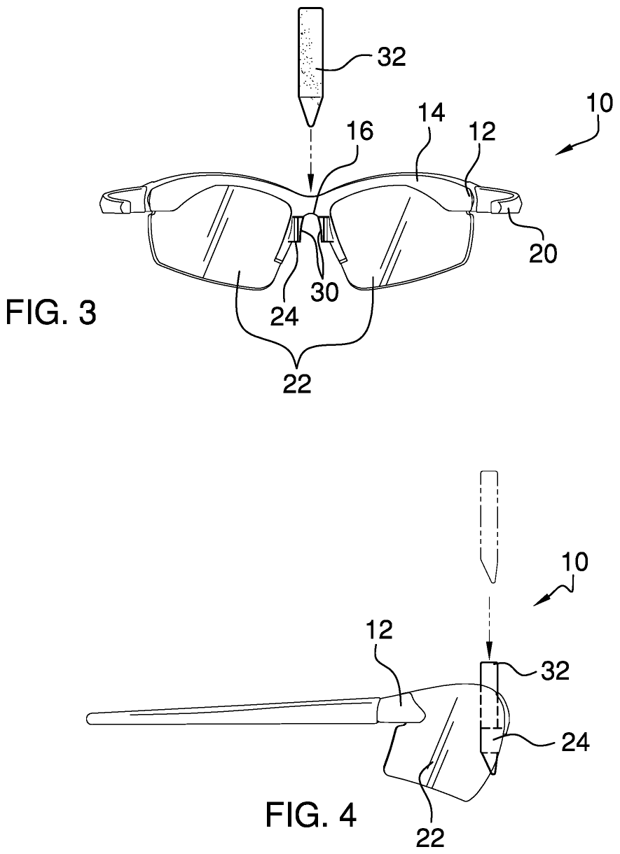

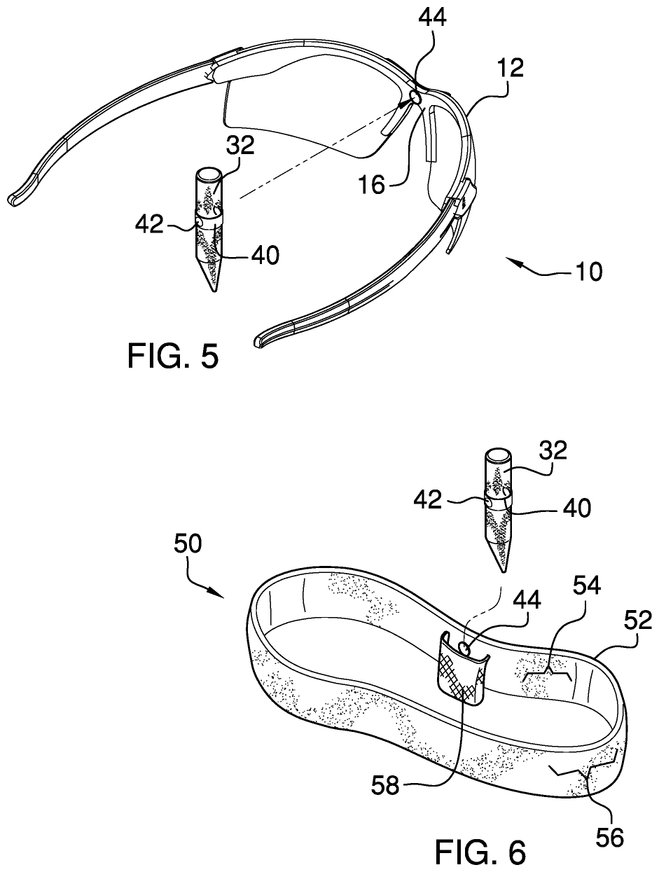

With reference now to the drawings, and in particular to FIGS. 1 through 6 thereof, a new aromatherapy device embodying the principles and concepts of an embodiment of the disclosure and generally designated by the reference numeral 10 will be described.

As best illustrated in FIGS. 1 through 6, the aromatherapy headwear apparatus 10 generally comprises a glasses frame 12 including a rim portion 14, a bridge portion 16 coupled to the rim portion 14, and a pair of arm portions 20 coupled to the rim portion 14. A pair of lenses 22 is coupled to the rim portion 14 of the glasses frame 12. The glasses frame 12 and the pair of lenses 22 may be a variety of styles including, but not limited to, safety glasses, reading glasses, and prescription glasses.

A diffuser holder 24 is coupled to an inner side 26 of the glasses frame 12. The diffuser holder 24 may include a pair of clips 28 coupled to the bridge portion 16 of the glasses frame to rest above the user's nose. Each clip 28 may have a curved inner face 30.

An aroma diffuser 32 is selectively engageable with the diffuser holder 24. The aroma diffuser 32 may have a cylindrical upper portion 36 and a tapered lower portion 38. The tapered lower portion 38 may be asymmetrical to accommodate the user's nose. The cylindrical upper portion 36 is pressingly engageable with the inner face 30 of the pair of clips. The aroma diffuser 32 may be a densely rolled absorbent fabric.

The diffuser holder 24 may include a ring 40 having a first magnet 42 coupled within the ring 40 and a second magnet 44. The second magnet 44 is coupled to the bridge portion 16 of the glasses frame. The first magnet 42 is selectively engageable with the second magnet 44 and the aroma diffuser 32 is slidingly engageable within the ring 40.

An alternative embodiment of the invention 50 generally comprises a headband 52 having a headband inside 54 and a headband outside 56. The diffuser holder 24 may include a pouch 58 coupled to the headband inside 54. The pouch 58 is porous and the aroma diffuser 32 is selectively engageable within the pouch 56. The second magnet 44 of the diffuser holder 24 may be coupled to the headband inside 54 adjacent the pouch 58 to additionally secure the diffuser holder 24 with the ring 40.

In use, the aroma diffuser 32 is loaded with a desired essential oil and engaged with the diffuser holder 24. The glasses frame 12 or the headband 52 are then worn by the user to place the aroma diffuser 32 on his or her forehead between the eyes to achieve a soothing effect during a procedure or before sleep.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An aromatherapy headwear apparatus comprising:
a glasses frame including a rim portion, a bridge portion coupled to the rim portion, and a pair of arm portions coupled to the rim portion;
a pair of lenses coupled to the glasses frame, the pair of lenses being coupled to the rim portion;
a diffuser holder coupled to an inner side of the glasses frame; and
an aroma diffuser being selectively engageable with the diffuser holder, the aroma diffuser holding and dispensing an essential oil.

2. The aromatherapy headwear apparatus of claim 1 further comprising the diffuser holder including a pair of clips coupled to the bridge portion of the glasses frame, the pair of clips pressingly securing the aroma diffuser.

3. The aromatherapy headwear apparatus of claim 1 further comprising the aroma diffuser having a cylindrical upper portion and a tapered lower portion.

4. The aromatherapy headwear apparatus of claim 3 further comprising the tapered lower portion being asymmetrical to accommodate a user's nose.

5. The aromatherapy headwear apparatus of claim 1 further comprising the diffuser holder including a ring having a first magnet and a second magnet, the second magnet being coupled to the bridge portion of the glasses frame, the first magnet being selectively engageable with the second magnet, the aroma diffuser being slidingly engageable within the ring.

6. The aromatherapy headwear apparatus of claim 1 further comprising the aroma diffuser being a densely rolled absorbent fabric.

7. An aromatherapy headwear apparatus comprising:
a glasses frame including a rim portion, a bridge portion coupled to the rim portion, and a pair of arm portions coupled to the rim portion;
a pair of lenses coupled to the glasses frame, the pair of lenses being coupled to the rim portion;
a diffuser holder coupled to an inner side of the glasses frame, the diffuser holder including a pair of clips coupled to the bridge portion of the glasses frame, each clip having a curved inner face; and
an aroma diffuser being selectively engageable with the diffuser holder, the aroma diffuser having a cylindrical upper portion and a tapered lower portion, the tapered lower portion being asymmetrical to accommodate a user's nose, the cylindrical upper portion pressingly engaging the inner face of the pair of clips, the aroma diffuser being a densely rolled absorbent fabric, the aroma diffuser holding and dispensing an essential oil.

8. An aromatherapy headwear apparatus comprising:
a headband having a headband inside and a headband outside;
a diffuser holder coupled to the headband inside; and
an aroma diffuser being selectively engageable with the diffuser holder, the aroma diffuser holding and dispensing an essential oil.

9. The aromatherapy headwear apparatus of claim 8 further comprising the aroma diffuser having a cylindrical upper portion and a tapered lower portion.

10. The aromatherapy headwear apparatus of claim 9 further comprising the tapered lower portion being asymmetrical to accommodate a user's nose.

11. The aromatherapy headwear apparatus of claim 8 further comprising the diffuser holder including a ring having a first magnet and a second magnet, the second magnet being coupled to the headband inside, the first magnet being selectively engageable with the second magnet, the aroma diffuser being slidingly engageable within the ring.

12. The aromatherapy headwear apparatus of claim 8 further comprising the diffuser holder including a pouch coupled to the headband inside, the pouch being porous, the aroma diffuser being selectively engageable within the pouch.
